(19) United States
(12) Patent Application Publication
(10) Pub. No.: US 2007/0077281 A1
(43) Pub. Date: Apr. 5, 2007

(54) Medical skin patches with a content of essential oils for treating colds, and processes for their production

(75) Inventors: Frank Theobald, Bad Breisig (DE); Wolfgang Laux, Diez (DE); Rene Eifler, Muelheim-Kaerlich (DE)

Correspondence Address: D. PETER HOCHBERG CO. L.P.A., 1940 EAST 6TH STREET, CLEVELAND, OH, 44114, US

(73) Assignee: LTS Lohmann Therapie-Systeme AG - A German Corporation, Andernach (DE)

(21) Appl. No.: 10/571,414

(22) PCT Filed: Sep. 9, 2004
(86) PCT No.: PCT/EP04/10046

§ 371 Date: Mar. 10, 2006

(30) Foreign Application Priority Data
Sep. 11, 2003 (DE) 103 41 933.0

(51) Int. Cl. A61F 13/02; A61L 15/16; A61K 36/61; A61K 36/53; A61K 36/534; A61K 36/73; A61K 36/13

(52) U.S. Cl. 424/448; 424/725; 424/745; 424/747; 424/765; 424/770; 424/742; 424/778

(57) ABSTRACT

Medical skin patches for treating colds by releasing essential oils through evaporation comprising a backing layer permeable to gas and water vapour and a hydrophile and pressure-sensitive adhesive polymer matrix connected to the backing layer. The matrix contains at least one essential oil, at least one hydrophile polymer, at least one substance having an adsorbent effect or/and at least one substance having an emulsifying effect, and at least one pressure-sensitive adhesive polymer. The water content of the matrix is less than 5% by weight, or even less than 1% by weight.

### CROSS-REFERENCE TO RELATED APPLICATIONS

This application is a National Stage application of International Application No. PCT/EP2004/010046, filed on Sep. 9, 2004, which claims priority of German application number 103 41 933.0, filed on Sep. 11, 2003.

### BACKGROUND OF THE INVENTION

1. Field of the Invention

The present invention relates to medical skin patches that enable the release of essential oils and are suitable for treating colds. The invention further relates to processes of production by which such patches can be obtained, as well as to the use of such patches for treating colds.

2. Description of the Prior Art

Colds involve obstruction of the respiratory passages, above all the nose and the bronchi, with coughing, hoarseness, bronchitis and other symptoms and are associated with more or less severe indisposition. Patients often get relief from inhaling essential oils. The action of these essential oils is due to the fact that they cause the mucus trapped in the nose or in the bronchi to loosen. In addition, these essential oils in part also possess antibiotic or disinfectant properties.

A disadvantage of inhalation is that it is relatively laborious and time-consuming since the essential oil has to be dispersed or dissolved in a hot water bath, and thereafter the hot water vapour, containing the volatile essential oils, has to be breathed in. Such a therapy is difficult to accomplish, for example, when travelling or in one's everyday professional life, all the more so since inhalation must be repeated several times a day.

Recently, various administration forms have been developed in an attempt to facilitate inhalation of essential oils in the treatment of colds. For example, there are preparations of ointments available on the market that contain essential oils and can be rubbed-in in the region of the chest, whereafter the essential oils are released due to the evaporation caused by the body heat and can be breathed in via the mouth and the nose. A disadvantage of this is that ointments are semi-solid, open preparations that must be applied directly to the patient's skin. Through sweating, e.g. as a result of fever, these semi-solid ointment preparations are gradually washed away from the skin or are absorbed by the person's clothing and thereby lose their efficacy. Hydrophobic ointment preparations are more resistant to sweat and water, it is true, but they cause an occlusion effect which may additionally raise the already elevated body temperature at the application site. In addition, application of an open, semi-solid preparation to the skin may subsequently cause the patient's clothes to become soiled.

U.S. Pat. No. 6,090,403 describes formulations wherein essential oils are contained in a hydrophile, pressure-sensitive adhesive preparation that is applied to a vapour-permeable solid support. However, when re-working the formulations described in U.S. Pat. No. 6,090,403, it was found that a high increase in viscosity occurs in these preparations already within a short time after manufacture, thus preventing further processing (e.g. coating) thereof. The maximum pot life, i.e. the processing period after the production of the preparation until the viscosity starts to increase, is only 1 to 2 hours. This is probably caused by the simultaneous presence of hydrophile polymers and aqueous components (e.g. water-containing glycerine or water-based latex polymer), which causes the hydrophile polymer to swell already during the production of the preparation. Since heavy swelling occurs during production, the water absorptivity and swellability of the formulations described in U.S. Pat. No. 6,090,403 are reduced.

In addition it has been found that phase separation occurs in these formulations between the hydrophile base polymer and the essential oil phase, which affects the reliability and durability of these medicinal preparations.

### SUMMARY OF THE INVENTION

The object of this invention therefore was to provide medicinal preparations that enable the release of essential oils for the treatment of colds and wherein the aforementioned disadvantages do not occur or are in any event considerably reduced.

This object is, surprisingly, achieved with a medical skin patch according to the present invention, as well as by the process of production according to the present invention.

### DETAILED DESCRIPTION OF THE PRESENT INVENTION

The skin patches according to the present invention comprise a backing layer permeable to gas and water vapour and a hydrophile polymer matrix connected with the backing layer and having pressure-sensitive adhesive properties. The polymer matrix contains:
at least one essential oil,
at least one hydrophile polymer,
at least one substance having an adsorbent effect or/and at least one substance having an emulsifying effect,
at least one pressure-sensitive adhesive polymer.

The water content of the matrix is less than 5% by weight, preferably less than 1% by weight.

The medical skin patches of the present invention are hydrophile, topic systems that are suitable for delivery of essential oils and for the treatment of colds. The essential oils are released from the polymer matrix of the patch as a result of the body heat and escape as vapours into the ambient air through the gas- and water-vapour-permeable backing layer and are subsequently inhaled via the patient's mouth and nose.

The essential oils are incorporated in a hydrophile, self-adhesive matrix which serves as a reservoir for these readily volatile oils and which, in the state of having been applied to the skin, is closed on the outward side by the above-mentioned backing layer so that soiling of clothing can be avoided. Due to their hydrophile character and the gas- and water vapour-permeable backing layer, these patches are well tolerated by the skin, and an occlusion effect is prevented.

The essential oil(s) contained in the hydrophile polymer matrix is/are preferably selected from the group comprising eucalyptol (cineol), menthol, thymol, borneol, bisabolol, mint oil, peppermint oil, spearmint oil, eucalyptus oil, camphor, turpentine oil, pine-needle oil, anise oil, fennel oil, thyme oil, rosemary oil, camomile oil and clove oil. Combinations of the aforementioned substances or mixtures of substances are also suitable; a combination of menthol, camphor and pine oil is especially preferred.

The overall proportion of the essential oil/oils is preferably 5 to 25% by weight, especially preferably in the range of 10 to 20% by weight, each relative to the said polymer matrix.

The medical skin patches according to the present invention contain at least one hydrophile polymer which, for reasons of manufacture, is present in a non-swollen state or is swollen only to a very low extent. In this context it is of essential importance that the water content of the hydrophile matrix is less than 5% by weight, preferably less than 1% by weight, during the manufacture as well as in the final product. Generally, the use of solvents, which would lead to swelling of the hydrophile polymers, must be largely avoided.

Due to the large proportions of non-swollen, hydrophile polymers in the matrix, the formulations according to the present invention are capable of absorbing very large amounts of moisture or water during the period in which they are applied on the skin, without losing their structural integrity and dropping off from the site of application.

The proportion of the hydrophile polymer/polymers is preferably in the range of from 15 to 50% by weight, especially preferably in the range of from 20-40% by weight, in each case relative to the said matrix.

Coming into consideration as hydrophile polymers are in principle those hydrophile polymers that possess good swelling properties and are compatible with essential oils and well tolerated by the skin.

The hydrophile polymer(s) is/are preferably selected from the group comprising cellulose derivatives, especially carboxymethyl cellulose, carboxypropyl cellulose, as well as polyvinyl alcohols, polyvinyl pyrrolidone, polyacrylic acid, polyacrylamide, polyethylene glycols, alginates, tragacanth, gums, especially karaya gum, acacia gum, guar gum, as well as xanthan, carrageenan, bentonite, starch and starch derivatives. Combinations of the aforementioned polymers may also be employed.

Another advantage of the inventive topical systems for release of essential oils consists in their having a cooling effect on the skin since the evaporation of the water and of the essential oils taking place above the water vapour-permeable backing layer leads to a cooling effect on the skin on account of the cold due to the evaporation.

Film or sheet materials, wovens (e.g. of polyester) or textile substances that exhibit these permeability properties may be used as the gas- and water vapour-permeable backing layer. Examples of suitable materials include open cell foamed plastics (e.g. polyurethane foam, polyethylene foam, plastic films rendered permeable by mechanical treatment, e.g. perforated polyethylene, polyethylene terephthalate and PVC films).

The skin patches according to the invention also contain at least one substance having an adsorbent effect or/and at least one substance having an emulsifying effect. Surprisingly, it has been found that by adding the substances it is possible, on the one hand, to prolong pot life (i.e. the time interval during which the matrix preparation containing the essential oils remains processible) and, on the other hand, to prevent the occurrence of phase separation between the hydrophile matrix polymer(s) and the essential oil phase.

Suitable as substances having an adsorbent effect are, in particular, the substances from the group comprising cyclodextrin and cyclodextrin derivatives, silicic acid and its derivatives (e.g. highly dispersed silicon dioxide, diatomaceous earth), as well as medicinal charcoal.

Suitable as substances having an emulsifying effect are, in particular, the following substances and groups of substances, either individually or in combination: sodium palmitate, sodium stearate, triethanolamine stearate, sodium lauryl sulfate, gum Arabic, alkonium bromide, benzalkonium bromide, cetylpyridium chloride, cetyl alcohol, stearyl alcohol, higher branched fatty alcohols, partial fatty acids of polyhydric alcohols, partial fatty acid esters of sorbitan, partial fatty acid esters of polyoxyethylene sorbitan, sorbitol ether of polyoxyethylene, fatty acid esters of polyoxyethylene, fatty alcohol ethers of polyoxyethylene, fatty acid esters of saccharose, fatty acid esters of polyglycerol, lecithin and complex emulsifiers such as, for example, complex-emulsifying cetyl stearyl alcohol. In addition, other emulsifiers known to those skilled in the art may be utilised.

The overall proportion of the substance(s) having an emulsifying effect or/and of the substance(s) having an adsorbent effect is preferably in the range of from 0.1 to 40% by weight, especially preferably in the range of from 1 to 30% by weight, and particularly in the range of from 5 to 20% by weight; in each case relative to the polymer matrix.

The hydrophile matrix of the skin patches according to the present invention exhibits pressure-sensitive adhesive properties. To this end, the matrix contains at least one pressure-sensitive adhesive polymer or a combination of two or more of such polymers.

The term “pressure-sensitive adhesive polymers” is in principle understood to mean those polymers which are contained in pressure-sensitive adhesive formulations and which are suitable for use on the skin.

For this purpose, polymers and combinations of polymers from the following group are particularly suitable: polyacrylates, polymethacrylates, polydimethylsiloxanes, polyvinyl acetate, polyisobutene, polyisobutylene, S—I—S block copolymers, polyterpenes, ethylene vinyl acetate copolymers, rubber and synthetic rubbers.

The proportion of the pressure-sensitive adhesive polymer/polymers is preferably 5 to 60% by weight, especially preferably 5 to 40% by weight, each relative to the polymer matrix.

According to a preferred embodiment, it is preferable for the pressure sensitive adhesive polymer(s) to be present in a crosslinked state. Crosslinking may be accomplished in a manner known to the skilled artisan, e.g. by using chemical agents (Al-acetylacetonate or Ti-acetylacetonate, in the case of polyacrylates) or by irradiation.

The hydrophile matrix containing the essential oils may in addition contain further formulation adjuvants, preferably moisturizers (e.g. anhydrous glycerol, propylene glycol or other polyhydric alcohols) or antifoaming agents. The proportion of the adjuvants may amount to 1 to 50% by weight, especially 5 to 30% by weight.

In a further preferred embodiment, the hydrophile matrix, following its production and during storage, is covered on its skin-contact side (on the side opposite the backing layer) with a detachable protective film. Suitable for this purpose are, for example, polyester or other plastics tolerated by the skin, such as polyvinyl chloride, ethylene vinyl acetate, vinyl acetate, polyethylene, polypropylene and cellulose derivatives, these films being made detachable by suitable surface treatment, such as siliconization.

The skin patches of the present invention are preferably welded in gas- and water vapour-tight packages.

The present invention further encompasses processes for the production of medical skin patches which comprise a hydrophile, pressure-sensitive adhesive polymer matrix with a content of at least one essential oil and which can be used for treating colds. More particularly the invention encompasses processes for the production of skin patches of the above described type. These processes comprise the following steps.

(a) Initially, a coating compound is produced by mixing the following components and possibly further optional components:
at least one essential oil,
at least one hydrophile polymer,
at least one pressure-sensitive adhesive polymer in a nonaqueous solvent,
at least one substance having an adsorbent effect or/and at least one substance having an emulsifying effect.

(b) This compound is coated onto a film (as a backing layer) that is permeable to gas and water vapour, as described above.

(c) By leaving this to dry or to solidify a pressure-sensitive adhesive, swellable polymer matrix is obtained.

(d) Subsequently, individual patches of the desired sizes can be obtained by punching out or cutting out. The pressure-sensitive adhesive surface of the patches may optionally be covered with a detachable protective layer prior to punching.

The water content of the coating compound should be less than 5% by weight, preferably less than 1% by weight. It is thereby achieved that the hydrophile polymer is not caused to swell or at most shows only the first signs of swelling.

The weight per unit area is preferably 20 to 400 g/m² (after drying). On drying, apart from the solvent(s) a part of the essential oil(s) is also evaporated; this must be taken into account by a corresponding addition to the recipe.

Examples of non-aqueous solvents which may be used include ethyl acetate, n-heptane, 2-propanol, ethanol or mixtures thereof, these solvents are particularly suited for pressure-sensitive polyacrylate adhesives and pressure-sensitive silicone adhesives. The selection of suitable solvents is above all dependent on the pressure-sensitive adhesive polymer(s); further suitable solvents are known to those skilled in the art.

It is furthermore advantageous to carry out the production of the preparations under cooling. In this case, at least step (a), or steps (a) and (b), is/are performed under cooling, preferably at temperatures below 15° C., particularly at temperatures below 10° C. It has been found that the heat input during mixing and homogenizing, too, may cause an undesirable increase in viscosity (by swelling of the hydrophile polymers) and a thickening of the coating compound.

The processes of the present invention are characterised in accordance with a preferred embodiment in that the coating compound produced in step (a) remains processible for a period of at least 3 hours, preferably at least 5 hours, and with particular preference for a period of at least 8 hours, following its production. It is thereby possible to prepare larger batches of compound and to process these into patches before an increase in viscosity occurs and processing is no longer possible.

Regarding the substances that are preferably taken into consideration for the individual components of the hydrophile polymer matrix (hydrophile polymers, pressure-sensitive adhesive polymers, substances having an adsorbent effect, substances having an emulsifying effect, essential oils and additional adjuvants) and their percentage proportions, reference is made to the particulars given hereinabove.

According to a preferred embodiment, the coating compound contains the following components:
30 to 40% by weight of polyacrylate pressure-sensitive adhesive,
0.1 to 1% by weight of Al-acetylacetonate,
20 to 40% by weight of hydrophile polymer(s), preferably karaya gum,
1 to 10% by weight of a substance/substances having an emulsifying effect, preferably polyoxyethylene sorbitan monooleate, such as TWEEN® 80,
0.5 to 10% by weight of antifoaming agent,
5 to 20% by weight of essential oil(s), preferably a combination of camphor, menthol and pine oil,
the sum of the proportions of the individual components always being 100% by weight.

According to a further preferred embodiment, the coating compound contains the following components:
5% to 10% by weight of polyacrylate pressure-sensitive adhesive,
20 to 35% by weight of glycerol (anhydrous)
15 to 25% by weight of propylene glycol
10 to 20% by weight of adsorbent substance(s), preferably a combination of silicic acid and hydroxypropyl-beta-cyclodextrin,
15 to 25% by weight of hydrophile polymer(s), preferably karaya gum,
5 to 20% by weight of essential oil(s), preferably a combination of camphor, menthol and pine oil,
   the sum of the proportions of the individual components always amounting to 100% by weight.

The invention will now be explained in more detail by means of the following examples of recipes.

### 1ST RECIPE EXAMPLE

| | |
|---|---|
| 36.2% by weight | of DUROTAK ® 387-2054 (polyacrylate pressure- |
| | sensitive adhesive; National Starch and Chemical |
| | Co.), |
| 0.5% by weight | of Al-acetylocetonate (crosslinking agent), |
| 36.7% by weight | of kereya gum, |
| 6.9% by weight | of TWEEN 80, |
| 6.9% by weight | of ATMOS ® 300 (antifoaming agent), |
| 6.2% by weight | of camphor, |
| 2.9% by weight | of menthol, |
| 3.7% by weight | of pine oil. |

### 2ND RECIPE EXAMPLE

| | |
|---|---|
| 19.0% bby weight | of karaya gum, |
| 29.0% by weight | of glycerol (anhydrous), |
| 19.5% by weight | of propylene glycol, |
| 7.0% by weight | of silic acid, |
| 6.5% by weight | of hydroxypropyl-beta-cyclodextrin, |
| 3.45% by weight | of menthol, |
| 3.8% bby weight | of pine oil, |
| 4.75% bby weight | of camphor, |
| 7.0% by weight | of DUROTAK ® 387-2287 (polyacrylate pressure- |
| | sensitive adhesive). |

The particulars relating to the pressure-sensitive adhesives employed refer only to the solid proportion of the pressure-sensitive adhesives present in solution.

The skin patches of the present invention can advantageously be used in methods for treating colds. In these methods a skin patch as described above or a skin patch produced according to a method described above is adhered to the diseased person's skin in the region of the chest, the back, the forehead, the neck or nape. In this way a continuous release of the said essential oils by evaporation is made possible, as well as the subsequent uptake of the evaporated essential oils via the person's nose or mouth by way of inhalation. In addition, these patches have a cooling effect on the skin caused by the cold due to evaporation. The patch is left on the site of application for a certain period of time, preferably 1 to 24 hours, and is thereafter removed and, if necessary, replaced by a new patch.

What has been described above are preferred aspects of the present invention. It is of course not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, combinations, modifications, and variations that fall within the spirit and scope of the appended claims.

1. A medicinal skin patch for the treatment of colds by releasing essential oils through evaporation, said skin patch comprising a backing layer permeable to gas and water vapour and a hydrophile and pressure-sensitive adhesive polymer matrix connected to said backing layer, said polymer matrix comprising:
at least one essential oil;
at least one hydrophile polymer;
at least one substance having an adsorbent effect or/and at least one substance having an emulsifying effect; and
at least one pressure-sensitive adhesive polymer,
the water content of said matrix being less than 5% by weight.

2. The skin patch according to claim 1, wherein the proportion of said at least one hydrophile polymer is 15 to 50% by weight relative to the said matrix.

3. The skin patch according to claim 1, wherein said polymer matrix contains at least one hydrophile polymer selected from the group consisting of cellulose derivatives, polyvinyl alcohols, polyvinyl pyrrolidone, polyacrylic acid, polyacrylamide, polyethylene glycols, alginates, tragacanth, gums, xanthan, carrageenan, bentonite, starch and starch derivatives.

4. The skin patch according to claim 1, wherein said at least one substance having an adsorbent effect is selected from the group consisting of cyclodextrins, cyclodextrin derivatives, silicic acid, silicic acid derivatives, and medicinal charcoal.

5. The skin patch according to claim 1, wherein said at least one substance having an emulsifying action is selected from the group consisting of sodium palmitate, sodium stearate, triethanolamine stearate, sodium lauryl sulfate, gum Arabic, alkonium bromide, benzalkonium bromide, cetylpyridium chloride, cetyl alcohol, stearyl alcohol, higher branched fatty alcohols, partial fatty acids of polyhydric alcohols, partial fatty acid esters of sorbitan, partial fatty acid esters of polyoxyethylene sorbitan, sorbitol ether of polyoxyethylene, fatty acid esters of polyoxyethylene, fatty alcohol ethers of polyoxyethylene, fatty acid esters of saccharose, fatty acid esters of polyglycerol, lecithin and complex emulsifiers.

6. The skin patch according to claim 1, wherein the overall proportion of said at least one substance having an emulsifying effect is 0.1 to 40% by weight, relative to said polymer matrix.

7. The skin patch according to claim 1, wherein said at least one essential oil is selected from the group consisting of eucalyptol (cineol), menthol, thymol, borneol, bisabolol, mint oil, peppermint oil, spearmint oil, eucalyptus oil, camphor, turpentine oil, pine-needle oil, anise oil, fennel oil, thyme oil, rosemary oil, camomile oil and clove oil.

8. The skin patch according to claim 1, wherein the overall proportion of said at least one essential oil is 5 to 25% by weight, relative to said polymer matrix.

9. The skin patch according to claim 1, wherein the proportion of said at least one pressure-sensitive adhesive polymer is 5 to 60% by weight, relative to said polymer matrix.

10. The skin patch according to claim 9, wherein said at least one pressure-sensitive adhesive polymer is selected from the group consisting of polyacrylates, polymethacrylates, polydimethyl siloxane, polyvinyl acetate, polyisobutene, polyisobutylene, S—I—S block copolymers, polyterpenes, ethylene vinyl acetate copolymers, rubber and synthetic rubbers.

11. The skin patch according to claim 1, wherein said polymer matrix contains additional adjuvants, and wherein the proportion of said adjuvants is 1 to 50% by weight.

12. The skin patch according to claim 1, wherein said polymer matrix includes a skin-facing surface, said skin-facing surface of the polymer matrix being covered with a detachable protective layer.

13. A process for the production of a medical skin patch comprising a backing layer permeable to gas and water vapour and a hydrophile, pressure-sensitive adhesive polymer matrix with a content of at least one essential oil for the treatment of colds, said process comprising the following steps:
(a) producing a coating compound containing a group of components by mixing said group of components, said group of components comprising:
at least one essential oil;
at least one hydrophile polymer;
at least one pressure-sensitive adhesive polymer in a nonaqueous solvent; and
at least one substance having an adsorbent effect or/and at least one substance having an emulsifying effect;
(b) coating said compound onto said backing layer permeable to gas and water vapour;
(c) leaving said backing layer to dry or solidify to obtain the polymer matrix; and
(d) punching out or cutting out individual patches.

14. The process according to claim 13, wherein at least step (a) is performed under cooling at temperatures below 15° C.

15. The process according to claim 13, wherein said coating compound produced in step (a) remains processible for a period of at least 3 hours following production.

16. The process according to claim 13, wherein the proportion of said at least one hydrophile polymer in the coating compound is 15 to 50% by weight.

17. The process according to claim 13, wherein the overall proportion of said at least one substance having an emulsifying effect or/and of said at least one substance having an adsorbent effect contained in the coating compound is 0.1 to 40% by weight.

18. The process according to claim 13, wherein the overall proportion of said at least one essential oil in the coating mass is 5 to 25% by weight.

19. The process according to claim 13, wherein the proportion of said at least one pressure-sensitive adhesive polymer in the coating compound is 5 to 60% by weight.

20. The process according to claim 13, further comprising the step of admixing additional adjuvants to the coating compound, the proportion of said adjuvants being 1 to 50% by weight.

21. The process according to claim 13, wherein said polymer matrix includes an adhesive surface, said adhesive surface of the polymer matrix being covered with a detachable protective layer.

22. The process according to claim 13, wherein said coating compound contains the following components:
30 to 40% by weight of polyacrylate pressure-sensitive adhesive solution;
0.1 to 1% by weight of Al-acetylacetonate;
20 to 40% by weight of said at least one hydrophile polymer;
1 to 10% by weight of said at least one substance having an emulsifying effect.
0.5 to 10% by weight of said antifoaming agent; and
5 to 20% by weight of said at least one essential oil;
the sum of the proportions of the individual components always being 100% by weight.

23. The process according to claim 13, wherein said coating compound contains the following components:
5% to 10% by weight of polyacrylate pressure-sensitive adhesive solution;
20 to 35% by weight of glycerol (anhydrous);
15 to 25% by weight of propylene glycol;
10 to 20% by weight of said at least one substance having an adsorbent effect;
15 to 25% by weight of said at least one hydrophile polymer; and
5 to 20% by weight of said at least one essential oil;
the sum of the proportions of the individual components always amounting to 100% by weight.

24. A method of treating colds, wherein a skin patch according to claim 1 or a skin patch produced according to the process of claim 13 is adhered to the diseased person's skin in the region of the chest, the back, the forehead, the neck or the nape for enabling a continuous release of essential oils by evaporation as well as the subsequent uptake of the evaporated essential oils by the person's nose or mouth by way of inhalation.

25. The skin patch according to claim 1, wherein the water content of said matrix is less than 1% by weight.

26. The skin patch according to claim 2, wherein the proportion of said at least one hydrophile polymer is 20-40% by weight relative to said matrix.

27. The patch according to claim 3, wherein said cellulose derivatives are selected from the group consisting of carboxymethyl cellulose and carboxypropyl cellulose and said gums are selected from the group consisting of karaya gum, acacia gum and guar gum.

28. The skin patch according to claim 6, wherein the overall proportion of said at least one substance having an emulsifying effect is 1 to 30% by weight relative to said polymer matrix.

29. The skin patch according to claim 28, wherein the overall proportion of said at least one substance having an emulsifying effect is 5 to 20% by weight relative to said polymer matrix.

30. The skin patch according to claim 7, wherein said at least one essential oil is a combination of menthol, camphor and pine oil.

31. The skin patch according to claim 8, wherein the overall proportion of said at least one essential oil is 10 to 20% by weight relative to said polymer matrix.

32. The skin patch according to claim 9, wherein the proportion of said at least one pressure-sensitive adhesive polymer is 5 to 40% by weight relative to said polymer matrix.

33. The skin patch according to claim 11, wherein said additional adjuvants are at least one of moisturizers and antifoaming agents, and wherein the proportion of said adjuvants is 5 to 30% by weight.

34. The process according to claim 14, wherein at least step (a) is performed at temperatures below 10° C.

35. The process according to claim 15, wherein said coating compound produced in step (a) remains processible for a period of at least 5 hours following production.

36. The process according to claim 35, wherein said coating compound produced in step (a) remains processible for a period of at least 8 hours following production.

37. The process according to claim 16, wherein the proportion of said at least one hydrophile polymer in the coating compound is 20-40% by weight.

38. The process according to claim 17, wherein the overall proportion of said at least one substance having an emulsifying effect or/and of said at least one substance having an adsorbent effect contained in the coating compound is 1 to 30% by weight

39. The process according to claim 38, wherein the overall proportion of said at least one substance having an emulsifying effect or/and of said at least one substance having an adsorbent effect contained in the coating compound is 5 to 20% by weight.

40. The process according to claim 18, wherein the overall proportion of said at least one essential oil in the coating mass is 10 to 20% by weight.

41. The process according to claim 19, wherein the proportion of said at least one pressure-sensitive adhesive polymer in the coating compound is 5 to 40% by weight.

42. The process according to claim 20, wherein said additional adjuvants are at least one of moisturizers and anti-foaming agents, the proportion of said adjuvants being 5 to 30% by weight.

43. The process according to claim 22, wherein said at least one hydrophile polymer is karaya gum, said at least one substance having an emulsifying effect is polyoxyethylene sorbitan monooleate, and said at least one essential oil is a combination of camphor, menthol and pine oil.

44. The process according to claim 23, wherein said at least one substance having an adsorbent effect; is a combination of silicic acid and hydroxypropyl-beta-cyclodextrin, said at least one hydrophile polymer is karaya gum, and said at least one essential oil is a combination of camphor, menthol and pine oil.
